(19) United States
(12) Patent Application Publication
(10) Pub. No.: US 2024/0116842 A1
(43) Pub. Date: Apr. 11, 2024

(54) MANUFACTURING OF ALLENE KETONES USING AN AMMONIUM (THIO)SULFATES OR HYDROGEN (THIO)SULFATES

(71) Applicant: DSM IP Assets B.V., HEERLEN (NL)

(72) Inventors: Fabrice AQUINO, Kaiseraugst (CH); Werner BONRATH, Kaiseraugst (CH); Francesco PACE, Kaiseraugst (CH)

(21) Appl. No.: 18/264,390

(22) PCT Filed: Feb. 7, 2022
(86) PCT No.: PCT/EP2022/052849

§ 371 Date: Aug. 4, 2023

(30) Foreign Application Priority Data
Feb. 8, 2021 (EP) 21155684.0

(51) Int. Cl. C07C 45/51; C07C 45/67

(52) U.S. Cl. CPC C07C 45/513; C07C 45/67

(57) ABSTRACT

The present invention relates to a process of manufacturing of allene ketone using specific ammonium (thio)sulfates or hydrogen (thio)sulfates as catalyst. The reaction provides the allene ketone in high yields and selectivities.

### TECHNICAL FIELD

The present invention relates to the manufacturing of allene ketones.

### BACKGROUND OF THE INVENTION

Allene ketones of the formula (I) are an important class of industrial chemicals and are central products in the synthesis of vitamins and aroma ingredients. particularly important. One of the possible synthetic routes uses tertiary propargyl carbinols as starting products.

U.S. Pat. No. 3,029,287 and G. Saucy et al. disclose in HeIv. Chim. Acta 1967, 50(4), 1158-1167 discloses the condensation of a tertiary propargyl alcohol and a ketal or an enol ether to form an allenic ketone in the presence of strong acids, particularly sulfuric acid or phosphoric acid or p-toluenesulfonic acid.

U.S. Pat. No. 6,380,437 discloses the condensation of a tertiary propargyl alcohol and a ketal or an alkenyl alkyl ether to form an allenic ketone in the presence of an aliphatic sulfonic acid or a metal salt thereof.

U.S. Pat. No. 3,330,867 and WO 2017/131607 disclose that an allene ketone can be isomerized to a unsaturated ketone with hydrogen.

The use of strong acid in the preparation of allene ketones is disadvantageous as these chemicals are hazardous in the handling and use special protection methods and require specific and corrosion-resistant materials for the equipment used in the manufacturing process.

### SUMMARY OF THE INVENTION

Therefore, the problem to be solved by the present invention is to offer a process of manufacturing of allene ketone of the formula (I) in high yield under the absence of strong acids and corrosive conditions.

Surprisingly, it has been found that a process according to the claim 1 and the reaction mixture according to claim 15 are able to solve this problem.

It has been found that specific ammonium (thio)sulfates or hydrogen (thio)sulfates are particularly well suited as catalyst for said reaction.

Further aspects of the invention are subject of further independent claims. Particularly preferred embodiments are subject of dependent claims.

### DETAILED DESCRIPTION OF THE INVENTION

In a first aspect, the present invention relates to a process for the manufacture of an allene ketone of the formula (I)

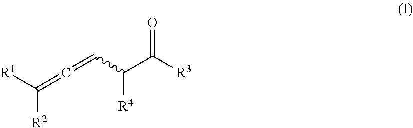

by the reaction of a compound of the formula (II) with a compound of the formula (111a) or (111b)

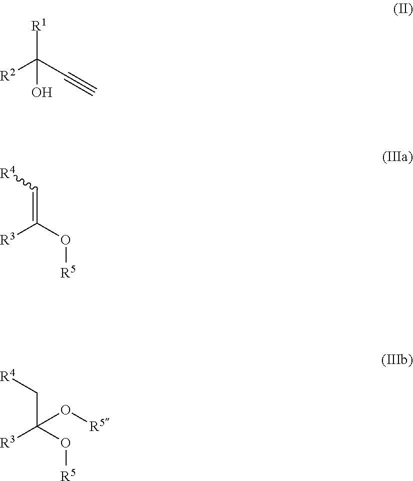

in the presence of an ammonium catalyst of the formula

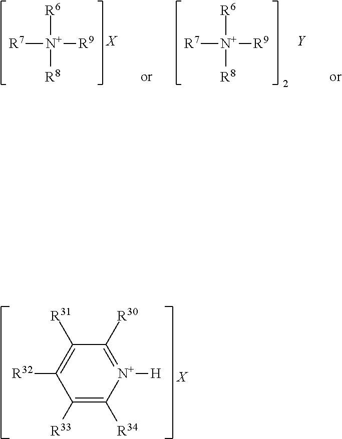

wherein
R¹ represents a methyl or ethyl group;
R² represents a saturated or unsaturated linear or branched or cyclic hydrocarbyl group with 1 to 46 C atoms;
R³ represents a methyl or an ethyl group;
R⁴ represents H or methyl or an ethyl group;
R⁵ represents a linear or branched C₁₋₁₀-alkyl group, particularly a methyl or an ethyl group;
R^{5′} and
R^{5″} represent
   either a linear or branched Ci_io-alkyl group, particularly a methyl or an ethyl group;
   or R^{5′} and R^{5″} form together a linear or branched C₁₋₁₀-alkylene group, particularly an ethylene or propylene group;
and
R⁶ and R⁷ and R⁸ and R⁹ represent independently from each other either H or a linear or branched C₁₋₁₀-alkyl group;
R³⁰, R³¹, R³², R³³ and R³⁴ represent independently from each other either H or a linear or branched C₁₋₁₂-alkyl or cycloalkyl group.
and
X=[HSO₄]⁻ or [HS₂O₃]⁻;
Y=[SO₄]²⁻ or [S₂O₃]²⁻; and
wherein the wavy line represents a carbon-carbon bond which when linked to the carbon-carbon double bond is either in the Z or in the E-configuration.

For sake of clarity, some terms used in the present document are defined as follows:

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

In case identical labels for symbols or groups are present in several formulae, in the present document, the definition of said group or symbol made in the context of one specific formula applies also to other formulae which comprises the same said label.

The term “independently from each other” in this document means, in the context of substituents, moieties, or groups, that identically designated substituents, moieties, or groups can occur simultaneously with a different meaning in the same molecule.

The term “(thio)sulfate” or “hydrogen (thio)sulfate”, respectively, embrace sulfate (═[SO₄]²⁻) as well as thiosulfate (═[S₂O₃]²⁻) or hydrogen sulfate (═[HSO₄]⁻) as well as hydrogen thiosulfate (═[HS₂O₃]⁻), respectively.

In the present document, any dotted line in formulae represents the bond by which a substituent is bound to the rest of a molecule.

In the present document, any wavy line represents independently from each other a carbon-carbon bond which when linked to the carbon-carbon double bond is either in the Z or in the E-configuration.

Compound of the formula (II) is reacted either with a compound of the formula (IIIa) or of the formula (IIIb) in the presence of a the specific ammonium (thio)sulfate or hydrogen (thio)sulfate catalyst (=“Cat”) as discussed later-on in great detail:

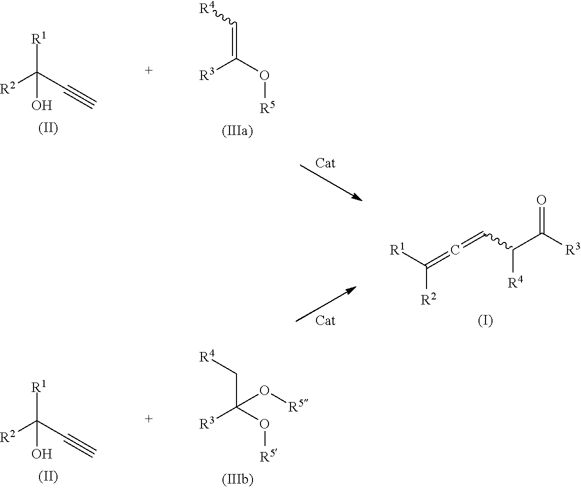

### Compound of the Formula (II)

The compounds of the formula (II) are substances known to the person skilled in the art.

R¹ represents a methyl or ethyl group, preferably a methyl group.

R² represents a saturated or unsaturated linear or branched or cyclic hydrocarbyl group with 1 to 46 C atoms, preferably a methyl group.

In a preferred embodiment, R² represents a group which is selected from the group consisting of the formula (R2-I), (R2-II), (R2-11I) and (R2-IV)

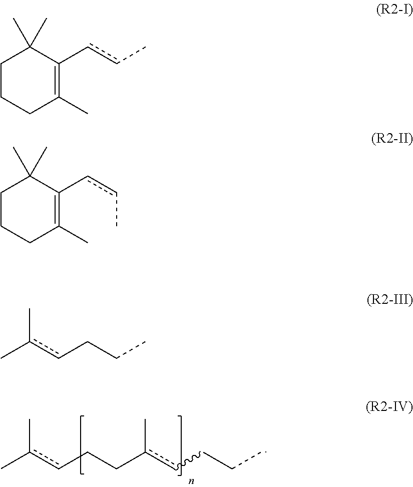

The dotted line represents the bond by which the substituent of the formula (R2-I), (R2-II), (R2-11I) or (R2-IV) is bound to the rest of the compound of the formula (I) or formula (II). Any double bond having dotted line ( [symbol] ) represents independently from each other either a single carbon-carbon bond or a double carbon-carbon bond. Any wavy line represents independently from each other a carbon-carbon bond which when linked to the carbon-carbon double bond is either in the Z or in the E-configuration.

In the above formulae n represents 1, 2, 3 or 4, particularly 1 or 2.

In one of the preferred embodiments, R² represents either a group of the formula (R2-I) or of the formula (R2-II).

In another preferred embodiment, R 2 represents either a group of the formula (R2-III) or of the formula (R2-IV).

It is preferred that compound of the formula (II) is selected from the group consisting of 2-methylbut-3-yn-2-ol (=methylbutynol, “MBY”), 3-methylpent-1-yn-3-ol (=ethylbutynol, “EBY”), 3,5-dimethylhex-1-yn-3-ol, 3,7-dimethyloct-6-en-1-yn-3-ol (=dehydrolinalool, “DLL”), 3,7-dimethyloct-1-yn-3-ol, 3,7-dimethylnon-6-en-1-yn-3-ol (=ethyldehydrolinalool, “EDLL”), 3,7,11-trimethyldodec-1-yn-3-ol, 3,7,11-trimethyldodec-6-en-1-yn-3-ol, 3,7,11-trimethyldodeca-6,10-dien-1-yn-3-ol (=de- hydronerolidol, “DNL”), 3,7,11-trimethyldodeca-4,6,10-trien-1-yn-3-ol (=ethinyl-pseudoionol, “EPI”), 3-methyl-5-(2,6,6-trimethylcyclohex-1 -en-1 -yl)pent-1 -yn-3-ol and 3-methyl-1 -(2,6,6-trimethylcyclohex-1 -en-1 -Apent-1 -en-4-yn-3-ol.

It is particularly preferred that compound of the formula (II) is selected from the group consisting of 2-methylbut-3-yn-2-ol (=methylbutynol, “MBY”), 3-methylpent-1-yn-3-ol (=ethylbutynol, “EBY”), 3,7-dimethyloct-6-en-1-yn-3-ol (=dehydrolinalool, “DLL”), 3,7-dimethylnon-6-en-1-yn-3-ol (=ethyldehydrolinalool, “EDLL”), 3,7,11-trimethyldodeca-6,10-dien-1-yn-3-ol (=dehydronerolidol, “DNL”) and 3,7,11-trimethyldodec-1-yn-3-ol.

It is even more preferred that the compound of the formula (II) is 3,7-dimethyloct-6-en-1-yn-3-ol (=dehydrolinalool, “DLL”) or 3,7,11-trimethyldodec-1-yn-3-ol.

### Compound of the Formula (IIIa)

The compounds of the formula (111a) are substances known to the person skilled in the art.

In formula (111a) R³ represents a methyl or an ethyl group and R⁴ represents H or methyl or an ethyl group and R 5 represents R 5 represents a linear or branched C₁₋₁₀-alkyl group, particularly a methyl or an ethyl group.

Preferably, the group R³ represents a methyl group.

Preferably, the group R⁴ represents H. Preferably, the group R⁵ represents a methyl group.

The compound of the formula (IIIa) is most preferably either isopropenyl methyl ether (“IPM”) or isopropenyl ethyl ether (“IPE”), particularly isopropenyl methyl ether (“IPM”).

Due to the synthesis of compound of the formula (IIIa), very often also mixtures of compounds of the formula (IIIa) are used for the reaction with compound of the formula (II). For example, for butenyl methyl ether, often a mixture of 2-methoxybut-1-ene and (E)-2-methoxybut-2-ene and (Z)-2-methoxybut-2-ene being prepared from methanol and methyl ethyl ketone, is used.

### Compound of the Formula (IIIb)

The compounds of the formula (IIIb) are substances known to the person skilled in the art.

In formula (IIIb) R³ represents a methyl or an ethyl group and R⁴ represents H or methyl or an ethyl group.

R^{5′} and R^{5″} represent in one embodiment each either a linear or branched C₁₋₁₀-alkyl group, particularly a methyl or an ethyl group. In another embodiment, R^{5′} and R^{5″} form together a linear or branched C₁₋₁₀-alkylene group, particularly an ethylene or propylene group.

Preferably, the group R³ represents a methyl group.

Preferably, the group R⁴ represents H.

In one preferred embodiment, R^{5′}=R^{5″} and particularly R^{5′}=R^{5″}=methyl or ethyl, more preferably R^{5′}=R^{5″}=CH₃.

In another preferred embodiment, R^{5′} and R^{5″} form together an ethylene (CH₂CH₂) or propylene (CH₂CH₂CH₂ or CH(CH₃)CH₂) group.

The compound of the formula (111b) is most preferably either 2,2-dimeth-oxypropane or 2,2-diethoxypropane or 2,2-dimethyl-1,3-dioxolane or 2,2,4-tri- methyl-1,3-dioxolane or 2,2-dimethyl-1,3-dioxane.

The compound of the formula (IIIb) is most preferably either 2,2-dimeth- oxypropane or 2,2-diethoxypropane, particularly 2,2-dimethoxypropane.

The use of the compound of formula (IIIa) is preferred over the compound of the formula (IIIb).

### Ammonium (thio)sulfates or Hydrogen (thio)sulfates

The reaction of a compound of the formula (II) with a compound of the formula (IIIa) or (IIIb) is performed in the presence of an ammonium catalyst of the formula

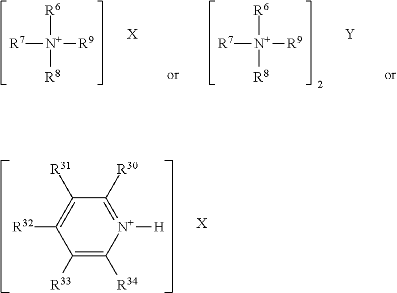

wherein
R⁶ and R⁷ and R⁸ and R⁹ represent independently from each other either H or a linear or branched C₁₋₁₀-alkyl group;
R³⁰, R³¹, R³², R³³ and R³⁴ represent independently from each other either H or a linear or branched C₁₋₁₂-alkyl or cycloalkyl group.
and
X=[HSO₄]⁻ or [HS₂O₃]⁻;
Y=[SO₄]²⁻ or [S₂O₃]²⁻; and

In other words, the ammonium catalyst suitable for the reaction of the compounds of the formula (II) with the compound of the formula (IIIa) or (IIIb) according to the present invention is a very specific ammonium compound in respect to the selection of cation but also of the anion.
It is preferred that that R⁶=R⁷=R⁸.
It is further preferred that R⁶=R⁷=R⁸=R⁹.
It is further preferred that R⁹=H.

So in one very preferred embodiment, the cation is the inorganic ammonium cation, i.e. NH₄⁺.

In another very preferred embodiment, the cation is a protonated tertiary amine, particularly triethylammonium and tributylammonium, preferred triethylammonium.

In another very preferred embodiment, the cation is a pyridinium or a pyridinium which is substituted by at least one linear or branched C₁₋₁₂-alkyl or cycloalkyl group.

It is preferred that R³⁰, R³¹, R³², R³³ and R³⁴ represent independently from each other either H or a methyl group

In one of these preferred embodiments, one or two groups of R³⁰, R³¹, R³², R³³ and R³⁴ represent a methyl group. Particularly preferred in this embodiment are α-picolinium, β-picolinium and γ-picolinium.

Mostly preferred of theses embodiments is pyridinium, i.e. R³⁰=R³¹=R³²=R³³=R³⁴=H.

The anion of the ammonium catalyst is either a sulfate (=[SO₄]²⁻) or a thio-sulfate (=[S²O₃]²⁻) or a hydrogen sulfate (=[HSO₄]⁻) or a hydrogen thiosulfate (=[HS₂O₃]⁻).

It has been found that the reaction is preferably performed when the molar ratio of the compound of the formula (II) to the compound of the formula (IIIa) or (IIIb) is ranging from 1:15 to 1:1.

In case of using the compound of the formula (IIIa), said ratio is more preferred in the range from 1:5 to 1:2, more preferably ranging from 1:3.5 to 1:2, most preferably ranging from 1:3 to 1:2, particularly ranging from 1:2.5 to 1:2.

In case of use of the compound of the formula (IIIb) said ratio is more preferred in the range from 1:10 to 1:2, more preferably ranging from 1:8 to 1:3, most preferably ranging from 1:8 to 1:5.

Furthermore, it is preferred that the amount of the ammonium catalyst is ranging from 0.01-1 mol-%, preferably ranging from 0.02-0.6 mol-%, more preferably ranging from 0.05-0.6 mol-%, based on the amount of the compound of the formula (II). It has been found that particularly for the ammonium catalyst of the formula

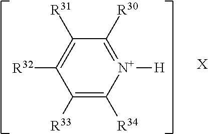

very small amount, in the range of 0.01 0.1 mol-%, preferably 0.01-0.05 mol-%, show particularly high yield and selectivity.

It has also been observed that the yield and selectivity increase when the molecular weight of the compound of formula (II) is increased using said ammonium catalyst at such low catalyst concentration.

It has been, furthermore, found that the very short reaction times of typically less than 3 hours can be achieved, even with concentration of ammonium catalysts of less than 0.5 mol-%.

Most preferably, the reaction is performed using a reaction time of between 60 minutes and 110 minutes at a concentration of the ammonium catalyst of between 0.5 and 0.05 mol-% or using a reaction time of between 2 hours and 22 hours at a concentration of the ammonium catalyst of between 0.1 and 0.01 mol-%.

The reaction is preferably carried out at a temperature ranging from 70 to 170° C. In one embodiment, the temperature is preferably ranging from 110 to 160° C., most preferably at a temperature ranging from 115 to 150° C. This temperature range is particularly suitable for isopropenyl methyl ether as compound of the formula (IIIa).

In another embodiment, the temperature is preferably ranging from 75 to 100° C., most preferably at a temperature ranging from 80 to 95° C. This temperature range is particularly suitable for compounds of the formula (IIIa) which either have R³=R⁴=methyl or ethyl or have R³= ethyl, most particularly for butenyl methyl ether as compound of the formula (IIIa).

The reaction is in one embodiment preferably carried out at a pressure ranging from 5 to 20 bara, more preferably at a pressure ranging from 6 to 15 bara. This pressure range is particularly suitable for isopropenyl methyl ether as compound of the formula (IIIa).

The reaction is in another embodiment preferably carried out at ambient pressure (1 bara). This pressure is particularly suitable for butenyl methyl ether as compound of the formula (IIIa).

The reaction can be carried out without solvent or in the presence of an organic solvent. Preferably the reaction is carried out without solvent.

Even if the reaction is carried out in the absence of an organic solvent, the starting materials, the compounds of the formula (II) and (IIIa) or (IIIb) , as well as said ammonium catalyst may still be provided in an organic solvent. Thus, there may be an amount of organic solvent up to 10 weight-%, preferably an amount of organic solvent up to 5 weight-%, more preferably an amount of organic solvent up to 3 weight-%, based on the total weight of the reaction mixture.

If the reaction is carried out in an organic solvent, polar aprotic organic solvents such as aliphatic ketones as e.g. acetone are preferred.

It has been found that the above reaction provides the compound of the formula (I) in high conversion, yield and selectivity.

It has been found that particularly the compound of the formula (I) being selected from the group consisting of 6-methylhepta-4,5-dien-2-one, 6,10-di-methylundeca-4,5-dien-2-one, 6,10-dimethylundeca-4,5,9-trien-2-one, 6,10,14-tri-methylpentadeca-4,5,9,13-tetraen-2-one, 6,10,14-trimethylpentadeca-4,5,9-trien-5 2-one, 6,10,14-trimethylpentadeca-4,5,13-trien-2-one and 6,10,14-trimethylpenta-deca-4,5-dien-2-one can be preferably produced.

The reaction mixture itself, with or without an organic solvent, is also an object of the present invention. 10 Thus, the present invention relates in a further aspect to a reaction mixture comprising
i) a compound of the formula (II),

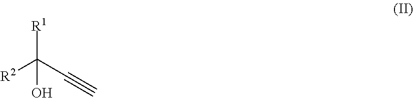

ii) a compound of the formula (IIIa) or (IIIb)

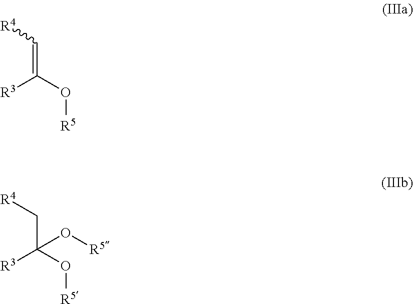

and
iii) an ammonium catalyst of the formula

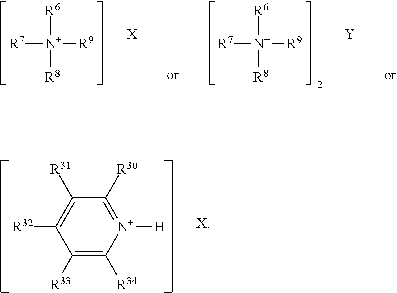

The compound of the formula (II) and of the formula (IIIa) or (IIIb) as well as the ammonium catalyst have been discussed to a great extend already above.

The compound of the formula (I) can be isomerized in the presence of a base or an acid, preferable a base, to a diene ketone of the formula (IV)

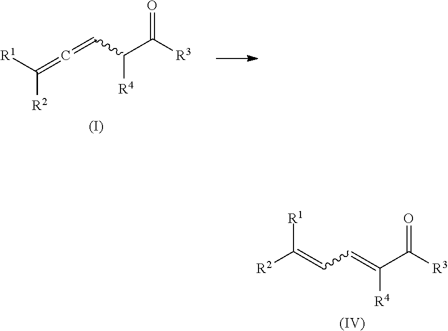

Thus, the present invention relates in a further aspect to a process for the manufacture of a diene ketone of the formula (IV)

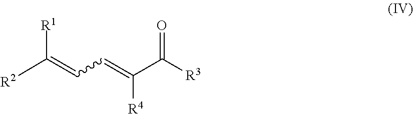

comprising the steps
a) preparing a compound of the formula (I) as discussed above already in great detail;
followed by
b) isomerization of compound of the formula (I) in the presence of a base or an acid, preferable a base, to yield the diene ketone of the formula (IV).

The isomerization of the compound of the formula (I) to the compound of the formula (IV) are principally known to the person skilled in the art, e.g. from U.S. Pat. No. 3,330,867 and WO 2017/131607, the entire content both of which is hereby incorporated by reference.

The base used in the isomerization step, i.e. step b) is preferably hydroxide or carbonate or hydrogen carbonate, preferably a hydroxide, of an alkali or earth alkali metal, particularly KOH or NaOH.

In another preferred embodiment, the base is a basic ion exchange resin, preferably the basic anion exchange resins Amberlite® IRA 900, Dowex® MSA-1, Diaion HPA25 or PA308 as well as Amberlysts® A260H, XE-4, XE-8, XE-8 new and XE-10 from DOW and equivalent resins with same chemical structure and similar physico-chemical properties.

The temperature in this isomerization step is preferably below 30° C., particularly between −10° C. and 25° C., preferably in the range of 0° C. to 10° C.

Said isomerization step is preferably performed in a C₁₋₆-alcohol, particularly in methanol, or in aqueous media.

Said processes allows the manufacturing of the compound of the formula (I) or (IV), respectively, in high yields and selectivities in respect to the compound of the formula (II). Hence, the present invention is offering big advantages over the processes known to person skilled in the art.

### Examples

The present invention is further illustrated by the following experiments.

### Experimental Series 1

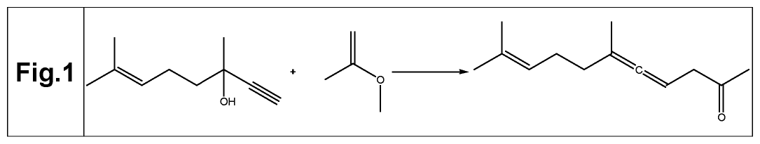

3,7-dimethyloct-6-en-1-yn-3-ol (=dehydrolinalool, “DLL”) was mixed with 4 equivalents of isopropenyl methyl ether (“IPM”) in the presence of the respective ammonium catalyst in the amount as given in table 1 and stirred at 115° C. during the reaction time as indicated in table 1. The respective product, i.e. 6,10-dimethylundeca-4,5,9-trien-2-one, was obtained in the yield and selectivity as indicated in table 1. (see FIG. 1)

TABLE 1
Formation of 6,10-dimethylundeca-4,5,9-trien-2-one from 3,7-dimethyloct-
6-en-1-yn-3-ol and isopropenyl methyl ether (DLL/IPM = ¼
[mol/mol]) using ammonium catalysts at 115° C. and pressure of 8.5 bara.

| | Ammonium | Amount² | Reaction | Yield | Selectivity |
|---|---|---|---|---|---|
| Example | catalyst¹ | [mol %] | time [min] | [%] | [%] |
| 1 | (pyH)HSO₄ | 0.1 | 100 | 96 | 96 |
| 2 | (NH₄)HSO₄ | 0.3 | 100 | 95 | 97 |
| 3 | (NH₄)HSO₄ | 0.1 | 100 | 93 | 93 |
| 4 | (NH₄)₂SO₄ | 0.2 | 100 | 81 | 91 |
| 5 | (NH₄)₂SO₄ | 0.1 | 100 | 62 | 86 |
| 6 | (Et₃NH)HSO₄ | 0.5 | 100 | 64 | 95 |
| 7 | (Et₃NH)HSO₄ | 0.1 | 100 | 43 | 95 |
| 8 | (NH₄)₂S₂O₃ | 1 | 100 | 58 | 100 |
| Ref. 1 | (pyH)H₂PO₄ | 0.5 | 100 | 35 | 54 |
| Ref. 2 | (pyH)H₃P₂O₇ | 0.1 | 100 | 24 | 41 |
| Ref. 3 | (Et₃NH)₂HPO₄ | 0.5 | 100 | 0 | n.a.³ |
| Ref. 4 | (NH₄)H₂PO₄ in H₂O | 1.0 | 100 | 3 | 15 |
| Ref. 5 | Na₂S₂O₃ | 0.5 | 100 | 0 | n.a.³ |

¹(Et₃NH) = triethylammonium; (pyH) = pyridinium;
²amount of catalyst relative to DLL
³n.a. = not applicable

The 6,10-dimethylundeca-4,5,9-trien-2-one from table 1 was quantitatively isomerized to 6,10-dimethylundeca-3,5,9-trien-2-one by the reaction as indicated in example 2 in WO 2011/131607.

### Experimental Series 2

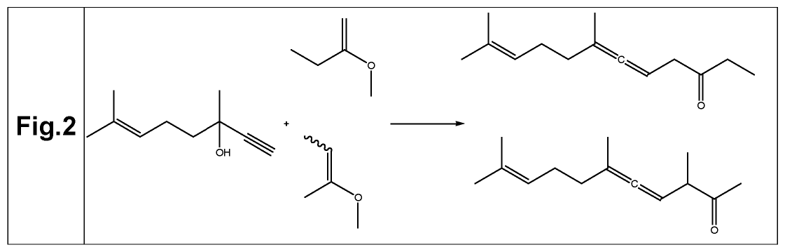

3,7-dimethyloct-6-en-1-yn-3-ol (=dehydrolinalool, “DLL”) was mixed with 2.6 equivalents of butenyl methyl ether (mixture of 2-methoxybut-1-ene/(E)-2-methoxybut-2-ene/(Z)-2-methoxybut-2-ene =53/37/10in the presence of the respective ammonium catalyst in the amount as given in table 2 and stirred at a temperature of 80-95° C. during the reaction time as indicated in table 2. The respective products, i.e. 7,11-dimethyldodeca-5,6,10-trien-3-one (=DMDTO) and 3,6,10-trimethylundeca-4,5,9-trien-2-one (=TMUTO), were obtained in the conversion, yield and selectivity as indicated in table 2 (see FIG. 2).

TABLE 2
Formation of 3,6,10-trimethylundeca-4,5,9-trien-2-one (=TMUTO) and 7,11-
dimethyldodeca-5,6,10-trien-3-one (DMDTO) from 3,7-dimethyloct-6-en-1-yn-3-ol
and butenyl methyl ether using ammonium catalysts and pressure of 1 bara.

| | Ammonium | Amount² | Reaction | Conversion | | Yield | Selectivity |
|---|---|---|---|---|---|---|---|
| Example | catalyst¹ | [mol %] | time [h] | [%] | TMUTO/DMDTO | [%] | [%] |
| 9 | (pyH)HSO₄ | 0.08 | 2.5 | 88.5 | 57/43 | 80.7 | 91 |
| 10 | (pyH)HSO₄ | 0.01 | 20 | 79.1 | 30/70 | 70.7 | 89 |
| 11 | (Et₃NH)HSO₄ | 0.5 | 20 | 64.0 | 41/59 | 36.4 | 57 |
| Ref. 6 | (Et₃NH)₂HPO₄ | 4 | 2.5 | 0 | n.a.³ | 0 | n.a.³ |
| Ref. 7 | (Et₃NH)₂HPO₄ | 0.5 | 20 | 4.4 | 56/44 | 0.9 | 20 |

¹(Et₃NH) = triethylammonium; (pyH) = pyridinium;
²amount of catalyst relative to DLL
³n.a. = not applicable

The 3,6,10-trimethylundeca-4,5,9-trien-2-one and 7,11-dimethyldodeca-5,6,10-trien-3-one from table 2 were quantitatively isomerized to 3,6,10-tri-methylundeca-3,5,9-trien-2-one, respectively 7,11-dimethyldodeca-4,6,10-trien-3-one, by the reaction as indicated in example 2 in WO 2011/131607.

### Experimental Series 3

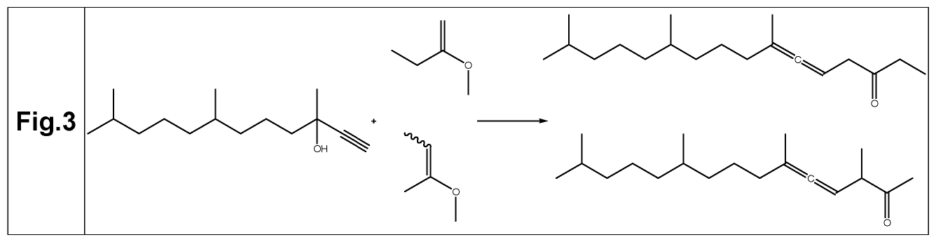

3,7,11-trimethyldodec-1-yn-3-ol was mixed with 2.6 equivalents of butenyl methyl ether (mixture of 2-methoxybut-1-ene/(E)-2-methoxybut-2-ene/(Z)-2-methoxybut-2-ene =53/37/10) in the presence of the respective ammonium catalyst in the amount as given in table 3 and stirred at a temperature of 80-95° C. during the reaction time as indicated in table 3. The respective products, i.e. 7,11,15-trimethylhexadeca-5,6-dien-3-one (TMHDO) and 3,6,10,14-tetramethylpentadeca-4,5-dien-2-one (TMPDO), were obtained in the conversion, yield and selectivity as indicated in table 3 (see FIG. 3).

TABLE 3
Formation of 7,11,15-trimethylhexadeca-5,6-dien-3-one (TMHDO) and 3,6,10,14-tetramethylpentadeca-
4,5-dien-2-one (TMPDO) from 3,7,11-trimethyldodec-1-yn-3-ol and butenyl methyl
ether using ammonium catalysts and pressure of 1 bara.

| | Ammonium | Amount² | Reaction | Conversion | | Yield | Selectivity |
|---|---|---|---|---|---|---|---|
| Example | catalyst¹ | [mol %] | time [h] | [%] | TMPDO/TMHDO | [%] | [%] |
| 9 | (pyH)HSO₄ | 0.08 | 2.5 | 97.0 | 62/38 | 93.8 | 97 |
| 10 | (pyH)HSO₄ | 0.01 | 20 | 79.7 | 30/70 | 76.1 | 96 |
| 11 | (Et₃NH)HSO₄ | 0.5 | 20 | 0.3 | n.a.³ | 0 | n.a.³ |
| Ref. 6 | (Et₃NH)₂HPO₄ | 4 | 2.5 | 0.1 | n.a.³ | 0 | n.a.³ |
| Ref. 7 | (Et₃NH)₂HPO₄ | 0.5 | 20 | 68.8 | 32/68 | 55.6 | 81 |

¹(Et₃NH) = triethylammonium; (pyH) = pyridinium;
²amount of catalyst relative to 3,7,11-trimethyldodec-1-yn-3-ol
³n.a. = not applicable

The 7,11,15-trimethylhexadeca-5,6-dien-3-one and 3,6,10,14-tetramethyl-pentadeca-4,5-dien-2-one from table 3 were quantitatively isomerized to 7,11,15-trimethylhexadeca-4,6-dien-3-one, respectively 3,6,10,14-tetramethylpentadeca-3,5-dien-2-one, by the reaction as indicated in example 2 in WO 2011/131607.

1. A process for the manufacture of an allene ketone of the formula (I) 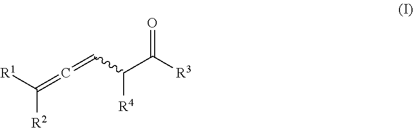
by the reaction of a compound of the formula (II) with a compound of the formula (IIIa) or (IIIb) 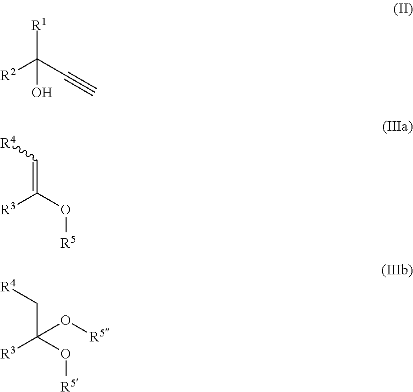
in the presence of an ammonium catalyst of the formula 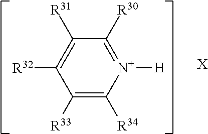
wherein
R¹ represents a methyl or ethyl group;
R² represents a saturated or unsaturated linear or branched or cyclic hydrocarbyl group with 1 to 46 C atoms;
R³ represents a methyl or an ethyl group;
R⁴ represents H or methyl or an ethyl group;
R⁵ represents a linear or branched C₁₋₁₀-alkyl group, particularly a methyl or an ethyl group;
R^{5′} and R^{5″} represent
either a linear or branched C₁₋₁₀-alkyl group, particularly a methyl or an ethyl group;
or R^{5′} and R^{5″} form together a linear or branched C₁₋₁₀-alkylene group, particularly an ethylene or propylene group;
and
R³⁰, R³¹, R³², R³³ and R³⁴ represent independently from each other either H or a linear or branched C₁₋₁₂-alkyl or cycloalkyl group.
and
X=[HSO₄]⁻ or [HS₂O₃]⁻;
wherein the wavy line represents a carbon-carbon bond which when linked to the carbon-carbon double bond is either in the Z or in the E-configuration.

2. The process according to claim 1, characterized in that R 1 represents a methyl group.

3. The process according to claim 1, characterized in that R³⁰, R³¹, R³², R³³ and R³⁴ represent independently from each other either H or a methyl group.

4. The process according to any of claim 1 characterized in one or two groups of
R³⁰, R³¹, R³², R³³ and R³⁴ represent a methyl group.

5. The process according to claim 1, characterized in that preferably R³⁰=R³¹=R³²=R³³=R³⁴H.

6. The process according to claim 1, characterized in that R²=methyl.

7. The process according to claim 1, characterized in that R 2 is selected from the group consisting of the formula (R2-1), (R2-11), (R2-11I) and (R2-IV), 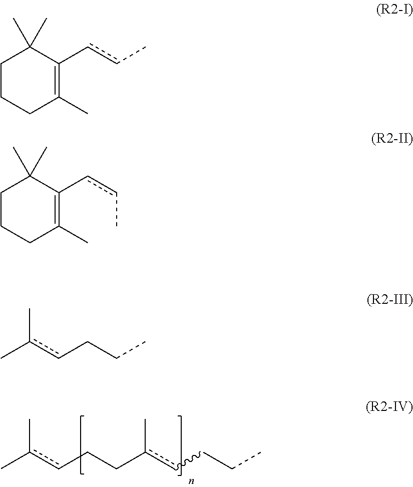
wherein the dotted line represents the bond by which the substituent of the formula (R2-1), (R2-11), (R2-11I) or (R2-IV) is bound to the rest of the compound of the formula (I) or the formula (II);
and wherein any double bond having dotted line ( [symbol] )represents independently from each other either a single carbon-carbon bond or a double carbon-carbon bond;
and wherein any wavy line represents independently from each other a carbon-carbon bond which when linked to the carbon-carbon double bond is either in the Z or in the E-configuration;
and wherein n represents 1, 2, 3 or 4, particularly 1 or 2.

8. The process according to claim 1, characterized in that the molar ratio of the compound of the formula (II) to the compound of the formula (IIIa) or (IIIb) is ranging from 1:15 to 1:1, and
in case of using the compound of the formula (IIIa)
said ratio is more preferred in the range from 1:5 to 1:2, more preferably ranging from 1:3.5 to 1:2, most preferably ranging from 1:3 to 1:2, particularly ranging from 1:2.5 to 1:2;
or in case of using the compound of the formula (IIIa)
said ratio is more preferred in the range from 1:10 to 1:2, more preferably ranging from 1:8 to 1:3, most preferably ranging from 1:8 to 1:5.

9. The process according to claim 1, characterized in that the amount of the ammonium catalyst is ranging from 0.01-1 mol-%, preferably ranging from 0.02-0.6 mol-%, more preferably ranging from 0.05-0.6 mol-%, based on the amount of the compound of the formula (II).

10. The process according to claim 1, characterized in that the compound of the general formula (I) is selected from the group consisting of 6-methylhepta-4,5-dien-2-one, 6,10-dimethylundeca-4,5-dien-2-one, 6,10-dimethylundeca-4,5,9-trien-2-one, 6,10,14-trimethylpentadeca-4,5,9,13-tetraen-2-one, 6,10,14-trimethylpentadeca-4,5,9-trien-2-one, 6,10,14-trimethylpentadeca-4,5,13-trien-2-one and 6,10,14-trimethylpentadeca-4,5-dien-2-one.

11. A process for the manufacture of a diene ketone of the formula (IV) 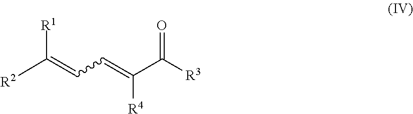
comprising the steps
a) preparing a compound of the formula (I) according to claim 1;
followed by
b) isomerization of compound of the formula (I) in the presence of a base or an acid, preferable a base, to yield the diene ketone of the formula (IV).

12. A reaction mixture comprising
i) a compound of the formula (II), 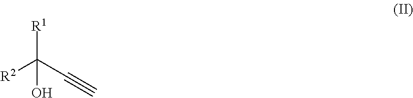
ii) a compound of the formula (IIIa) or (IIIb)
and 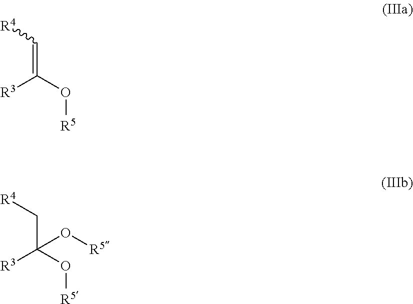
iii) an ammonium catalyst of the formula 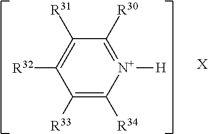
wherein
R¹ represents a methyl or ethyl group;
R² represents a saturated or unsaturated linear or branched or cyclic hydrocarbyl group with 1 to 46 C atoms;
R³ represents a methyl or an ethyl group;
R⁴ represents H or methyl or an ethyl group;
R⁵ represents a linear or branched C₁₋₁₀-alkyl group, particularly a methyl or an ethyl group;
R^{5′} and R^{5″} represent
either a linear or branched C₁₋₁₀-alkyl group, particularly a methyl or an ethyl group;
or R^{5′} and R^{5″} form together a linear or branched C₁₋₁₀-alkylene group, particularly an ethylene or propylene group;
and
X=[HSO₄]⁻ or [HS₂O₃]⁻;
and wherein any wavy line represents independently from each other a carbon-carbon bond which when linked to the carbon-carbon double bond is either in the Z or in the E-configuration.
